(19) United States
(12) Patent Application Publication
(10) Pub. No.: US 2022/0244120 A1
(43) Pub. Date: Aug. 4, 2022

(54) A BRACE FOR MANAGEMENT OF AN ANATOMICAL STRUCTURE; AND APPLICATIONS THEREOF

(71) Applicant: TMT SYSTEMS APS, København Ø (DK)

(72) Inventor: Steen ANDREASEN, København V (DK)

(73) Assignee: TMT SYSTEMS APS, København Ø (DK)

(21) Appl. No.: 17/624,935

(22) PCT Filed: Jul. 3, 2020
(86) PCT No.: PCT/EP2020/068885

§ 371 Date: Jan. 5, 2022

(30) Foreign Application Priority Data
Jul. 10, 2019 (DK) PA 2019 70455

(51) Int. Cl. G01L 5/00

(52) U.S. Cl. CPC G01L 5/0052

(57) ABSTRACT

Disclosed is a brace for management of an injured part of an anatomical structure. The brace comprises a proximal part extending from a proximal joint end towards a proximal free end along a proximal axis. The brace comprises a proximal fixation configured for supporting a proximal anatomical structure. The proximal part at the proximal joint end interacting via a brace joint with a distal part extending from a distal joint end towards an opposite a distal free end along a distal axis. The proximal part and the distal part are operatively connected via the brace joint. Also disclosed is a brace and a method of measuring a threshold injury force on an anatomical structure.

### FIELD

The present disclosure relates generally to devices moving and stabilizing parts of a limb across an anatomical joint, to allow immobilisation, instability testing and rehabilitation of anatomic structures after injury.

### BACKGROUND

Anatomic joints connect bones in the body and allow movement in the skeletal system. Injury to anatomic joints might involve bony structures, ligaments, muscle tears, tendon ruptures and more. Most injuries are stable and will heal without or with little compromise to the functional whole across the joint. Certain injuries are severe and cause instability across the anatomical joint, requiring: internal or external fixation (either through surgical stabilization or an external cast/brace); reduction (if the anatomic joint has been dislocated and requires realignment; rehabilitation to restore optimal range of motion across the affected joint.

### DESCRIPTION

An objective is achieved by a brace for management of an injured part of an anatomical structure. The brace comprises a proximal part extending from a proximal joint end towards a proximal free end along a proximal axis. The brace comprises a proximal fixation configured for supporting a proximal anatomical structure. The proximal part at the proximal joint end interacting via a brace joint with a distal part extending from a distal joint end towards an opposite a distal free end along a distal axis.

The proximal part and the distal part are operatively connected via the brace joint.

The distal part may comprise a distal plate operatively connected to a proximal plate via two distal parallel arms for a displacement of the proximal plate relative to the distal plate, such that the distal plate is displaced along a tilt axis perpendicular to both the proximal axis and the distal axis.

The brace may have a pair of proximal parallel arms connected to and arranged along the proximal part. The proximal parallel arms may be operatively connected to the distal part for a displacement of the distal part relative to the proximal part substantially along the distal axis.

The degree of instability is often negatively correlated to the functional outcome after an injury. Joint instability is not always apparent. When unstable injuries are suspected, the stability of the ligaments and other anatomic structures are tested using joint specific stress-tests, sometimes also referred to as a laxity tests, to assess whether stabilization is indicated. The brace as disclosed improves the stability across an injured joint.

Both joint reduction, external immobilization, stress testing and rehabilitation, is traditionally performed manually. Joint reductions performed in emergency departments across the world are often labour intensive and inadequate. The brace as disclosed mitigates such insensitivity and inadequacies.

Stress tests performed manually are highly subjective with a lack of normative values and standardization, causing a high rate of false positive findings overestimating instability. This can cause unindicated fixation, affecting post injury functional outcome. Manual rehabilitation of range of motion with a qualified physiotherapist is the gold standard. However, many patients receive limited time with their physiotherapists and do not perform exercises correctly and frequently enough to optimize post injury functional outcome. The brace as disclosed is a functional external fixation product allowing independent multi-planar movement and locking, facilitating reduction and immobilization, as well as standardized instability testing and continuous passive range of motion and resistance rehabilitation means in all required planes.

A person skilled in the art will have arranged the brace in relation to a given anatomical structure with a view to managing an injured part, such as an anatomical joint between say an anatomical proximal structure and an anatomical distal structure.

Thus, the distal part of the brace will engage with or be positioned along an anatomical distal structure and the proximal part of the brace will engage with or be positioned along an anatomical proximal structure. Operating the brace will then apply displacements and/or forces to the anatomical structure, e.g. the anatomical joint, which may be injured and in need of management.

In example, the brace may be applied to an ankle between a lower leg and a foot when the ankle is injured and in need of management.

In example, the brace may be applied to a proximal and distal phalangeal bone of a finger, if the interphalangeal joint is injured. As such, a person skilled in the art will appreciate the general principles exemplified in view of an ankle and be able to apply the same structural features to a brace for other anatomical structures.

By applying a 1^{st} activation means to the proximal parallel arms, the distal part of the brace will move along the distal axis relative to the proximal part.

In example, when operated with a foot fixed to the distal part and an under leg fixed to the proximal part, then all things equal the foot will be displaced and/or forces will be applied to the foot in the dorsal-ventral direction in the sagittal plane, that is back and forth, anterior-posterior.

A person skilled in the art will appreciate that deviations from anatomical references may result depending on the actual configuration according to operational conditions/positions of the brace. As is clear or as will be disclosed, the distal part may be in an angle or displaced by means other structural characteristics of the brace.

The proximal parallel arms provided will provide sufficient strength in forces and displacement as well as precise movements along the distal axis.

The proximal parallel arms will at one end be adjoined towards the proximal free end, which may be the superior direction. At the opposite end of the proximal parallel arms, which may be the inferior direction, the proximal parallel arm will be adjoined to the brace joint.

The arrangement of arms in a pair, e.g. two arms, of proximal parallel arms provides the arms that are shifted or displaced relative to each other along the distal axis.

The arrangement of arms may include more than a pair of arms, e.g. more than two arms. There may be multiple pairs of arms, or multiple or more than a pair of arms, e.g. three arms or four arms.

The ends of an arm may have a pivot joint part, which may be attached to a longitudinal part of the arm, i.e. a shaft of the arm. The arm may be a monotonic structure.

In an aspect there are two pairs of arms, e.g. four arms, each pair arranged in parallel to each other. For example, a first arm and a second arm are arranged parallel to each other and a third and a fourth arm are arranged parallel to each other. Additionally, the first arm and the third arm may be arranged parallel and the second and the fourth arm may be arranged parallel.

The 1^{st} activation means may be applied to the proximal parallel arms at the pivotal joints towards the proximal end.

The 1^{st} activation means may be applied to the distal part.

The parallel arms allow a near linear displacement along the distal axis, while providing multi-planar rigidity in the other directions across the injured anatomical structure.

In an aspect the brace joint is configured with locking means for locking the brace joint and placing the proximal part and the distal part in a fixed position relative to each other. In an aspect the brace joint is configured to receive gauge means for gauging a force on and/or a displacement of the proximal part and the distal part relative to each other.

The locking means may be a bolt-screw arrangement. The locking means may include recesses, or pins blocking the turn of blocking means. The locking means may include complementary faces being tightened together and optimally being secured. The gauge means may be a Newton-meter. The gauge means may be a torque wrench argument. The torque wrench may be integrated or applied separately. The gauge means may be a torque wrench or digital torque-meter. Such wrench may be applied to a bolt.

In an aspect, the gauge may be a spring element inserted into the brace to gauge the displacement. In an aspect there may be a scale to measure the displacement. The spring may be a linear spring.

The locking means and/or the gauge means may apply to the 1^{st} activation and/or any one or more of the following 2^{nd}-6^{th} activations. Likewise, scales as required, i.e. linear scales and/or angular scales may be arranged as required.

In an aspect, the proximal part supports a proximal free end fixation configured for supporting an adjacent anatomical structure. The proximal part may support a proximal guide being configured for a displacement of the distal part relative to the proximal free end fixation along the proximal axis. The displacement may be achieved by a 2^{nd} activation means.

Such activation results in a displacement of the proximal fixation. Consequently, when applied on an adjacent anatomical structure, e.g. an upper leg relative to a foot, the displacement is in a cranial-caudal direction.

Often severe injuries across anatomic structures are impacted by the force of the trauma, requiring distraction in a cranial-caudal direction.

The locking means may be as disclosed. The gauge means may be as disclosed.

In an aspect, the brace joint is configured to rotate the distal part in a plane spanned by the proximal axis and the distal axis. The rotation of the distal part may be relative to the proximal part, e.g. such that an angle between the distal part and the proximal part increases or decreases.

The rotation may be applied by a 3^{rd} activation means.

The rotation may be substantially a flexion or extension, e.g. causing a flexion or extension in a joint of the anatomical structure, or rotation in an associated sagittal plane.

When reducing dislocated anatomical joints, rotation is essential to align. Furthermore, many stress tests involve external and internal rotation of the distal part relative to the proximal part across an injured joint, testing potentially unstable anatomic structures. A rotational range of motion is the motion across the large joints such as the knee, hip, ankle, wrist and elbow and a main focus of rehabilitation.

The locking means may be as disclosed. The gauge means may be as disclosed.

In an aspect, the distal part comprises a distal plate operatively connected to a proximal plate via a pair of distal parallel arms for a displacement of the proximal plate relative to the distal plate.

By applying a 4^{th} activation means, the result is a displacement of the distal part in a medial-lateral direction. The distal part may be displaced in a medial-lateral direction relative to the proximal part.

The distal part may comprise a first plate arranged towards the proximal end, which plate is a proximal plate, and a second plate arranged opposite towards the distal end, which plate is a distal plate. The two plates are interconnected by a set of, e.g. two, distal parallel arms. The distal plate is connected to the brace joint. As such, operation of the brace joint results in operation of the distal plate and consequently the operation of the distal parallel arms will result in the proximal plate being displaced relative to the brace joint, the distal plate and the proximal part. Alternatively, the distal plate and the proximal plate may be interconnected by one arm.

In an aspect the proximal plate is a proximal side of an outer box containing an inner box that has a proximal side towards the proximal side of the outer box. The distal plate may be a side of the inner box. The outer box and inner box are operatively connected by the distal parallel arms. The inner box is connected to the brace joint. As such operation of the brace joint results in operation of the inner box and consequently the operation of the distal parallel arms will result in the outer box and thus the proximal plate being displaced relative to the brace joint, the inner box and the proximal part.

The main effect of the 4^{th} activation means is alignment medial and lateral dislocation of the distal part relative to the proximal part, but is also used in instability stress tests when assessing medial or lateral instability.

The locking means may be as disclosed. The gauge means may be as disclosed.

In an aspect, the brace joint is configured to rotate the distal part substantially about the proximal axis. The rotation of the distal part may be relative to the proximal part. The rotation of the distal part may be relative to the proximal part.

By applying a 5^{th} activation means to the brace joint or the distal part, the rotation is substantially about the cranial-caudal axis i.e. in a transverse plane.

The locking means may be as disclosed. The gauge means may be as disclosed.

In an aspect the brace joint is configured to rotate the distal part about the distal axis. The rotation of the distal part may be relative to the proximal part.

By applying a 6^{th} activation means to the brace joint or the distal part, the rotation is substantially about the dorsal-ventral axis.

The locking means may be as disclosed. The gauge means may be as disclosed.

The above-mentioned means for displacement and rotation may be applied alone, in combination or all together.

Each provides a precise and/or repeatable displacement or rotation which allows for improved and consistent management of an anatomical structure. In example an ankle may be managed within specific limits.

In a special aspect, all activations 1^{st}-6^{th} are implemented and the brace will displace/rotate in a total of six-degrees of freedom. The displacement/rotation may be as a route wherein the order of activation is a part. Thereby the brace allows for a wide range of managements, including complex managements.

In an example the brace is configured for management of an injured, unstable ankle.

The proximal part is attached to the lower leg and above the knee. The distal part is attached to the foot in a rigid interphase using a fastener, such as an adjustable casing and straps.

Reduction: If the injury requires reduction, posterior or anterior ankle displacement can be addressed by activating the 1^{st} activation means, impaction and shortening can be addressed by activation the 2nd activation means and the ankle injury can be distracted. The ankle can be placed into neutral or dorsal flexion by activating the 3^{rd} activation means. If residual medial gapping is apparent, the lateral translation can be addressed by activating the 4^{th} activation means. Unstable ankle injuries tend to be externally rotated, and through activating the 5^{th} activation means internal rotation can be applied. Lastly, the ankle can tilt when grossly unstable which can be addressed with the 6^{th} activation means. The 4^{th} activation facilitates enhanced reduction treatment of a joint, to reposition the distal anatomical structure medially or laterally relative to the proximal anatomical structure, especially in reduction treatment of for example an ankle fracture or dislocation, which typically happens in medially or laterally.

Immobilization: All positions can be locked independently when the position is judged to be correct. If further reduction is necessary, each activation means can be unlocked and activated again independently. The device can as such immobilize severely unstable injuries until surgery if the skin interphase does not harm the skin. In a different setting, a moldable cast sock is applied under the device, and is chemically activated and hardened after reduction, allowing the device to be removed, with the cast and thus the ankle remaining in the correct position. The device is constructed from a radiolucent material allowing both reduction and instability testing to be performed under continuous radiography (referred to as fluoroscopy), which allows visualization of mainly bony structures within the ankle joint.

Instability testing of the ankle: Activating the 1^{st} activation means through a standardized torque-meter will test the anatomical structures securing anterior/posterior stability, mainly the syndesmosis, the posterior malleolus, the talar dome and ligaments. Activating the 4^{th} activation means with a known force will test the lateral anatomical structures, which are the most common to sustain injury in ankle trauma. Externally rotating the foot through the 5^{th} activation means with a known force will test the syndesmosis, a ligamentous structure between the fibula and tibia, the two bones constituting the lower leg, and a pivotal anatomical structure securing stability of the ankle joint. Activating the 6^{th} activation means will further test the lateral and medial anatomical structures.

Rehabilitation: By locking all undesired movements, the ankle can be specifically activated in the desired plane, either by the patient's own force in active motion or passively through a motor moving the joint in continuous passive motion, applying force to the 1st-6^{th} activation means. If more or all directional movements are desired, the activation means can be unlocked accordingly. A desired resistance can be applied to each activation means individually. Range of motion, force and resistance can be measured and tracked over time by the patient, therapist or a specific mobile/computer application.

In an aspect the brace is made of carbon fiber, plastic material and/or other material in order to be radiolucent to radiography. Continuous radiography (referred to as fluoroscopy) is often used in the emergency department or in the operating theater to visualize mainly bony structures and secure correct alignment during reduction. Thus, it is important for the brace to allow full radiographical visualization of the affected anatomical structure.

An objective may be achieved by a brace for injuring an anatomical structure of a human cadaver. Such brace for injuring an anatomical structure of a human cadaver may be denoted a trauma brace. The brace as disclosed herein may be used for injuring an anatomical structure of a human cadaver. Hence, the disclosed brace may be a trauma brace. The brace for injuring an anatomical structure may be a more rigid or stiff version of the brace as disclosed.

Structural elements may be changed to stronger materials or stiffer materials. Materials may be selected according to elastic modulus, E, a starting point.

| Material | E [Gpa] |
|---|---|
| E_{peek carbon 50}: | 52 |
| E_{carbon fiber}: | 130 |
| Eₐₗᵤₘᵢₙᵢᵤₘ: | 70 |
| E_{stainless steel}: | 200 |

In example, peek carbon peek 50 maybe be changed to aluminum. Peek carbon 50 may be changed to stainless steel. Carbon fiber may be changed to stainless steel. Aluminum may be changed to carbon fiber.

In example, peek carbon components can be changed to aluminum or stainless steel with an estimated stiffness increase from 30% to 270%, respectively.

Carbon fiber components can be changed to stainless steel with an estimated stiffness increase of 54%.

Knowing typical forces needed to provide an injury to a joint, or anatomical structure of a give type, the above material characteristics provide a starting point to select the proper and required strength:

As a starting point for a brace for inducing a trauma on an ankle, substantial multi-planar forces need to be applied to the anatomical structures within the ankle and the brace will be a stainless steel construct to allow maximal rigidity.

A person skilled in the art will appreciate that a brace for a finger is different in size and may be different with respect to material compared to a brace for an ankle. Using the above guidelines and principles a person skilled in the art will be able to device a brace for a finger, a wrist, an elbow, a knee etc.

A person skilled in the art will appreciate the need for a fastener, such as one or more straps or alike, to fixation of the anatomical structure to the brace. The fasteners may be attached to the proximal free end fixation or the proximal fixation. Fasteners may also be attached to the distal part, such as to the distal plate.

The brace may be arranged on a support, which may be a plate or a table.

An objective may be achieved by a method for measuring a threshold injury force on an anatomical structure of a human cadaver. The method may comprise one or more acts as follows.

There is an act of providing a human cadaver having an anatomical structure.

There is an act of arranging a brace about an anatomical structure.

There is an act of applying one or more forces to the anatomical structure by use of the brace until a point of injury of an anatomical structure.

There is an act of measuring the one or more forces and/or displacements, or both, until and/or at the point of injury.

Thereby is achieved a way of tabulating forces, displacements leading to an injury.

The methodology will provide standardized tabulation.

In an aspect the brace is as disclosed herein. The brace may be implemented with one, more or all of the 1^{st} to 6^{th} activation means applied to the respective parts of the brace.

In example, a certain torque, T, is needed to break, i.e. injure, an ankle.

The required torque will depend on the species and in the case of a human, the person being man, a woman, a child, and the individual size.

A handle, here of a length from the point of activation on the brace to the point of applying a force may be applied the activation means. The handle may be chosen according to the following appliance of a weight m for a number of lengths 1. For l=50 cm; m=10 kg then: T=50 Nm. For l=50 cm; m=20 kg then: T=100 Nm. For l=50 cm; m=30 kg then: T=150 Nm. For l=50 cm; m=40 kg then: T=200 Nm. The handle may be configured to be removably attached.

To break an ankle, a brace may be configured with following displacement, including rotations, ranges.

The brace may be configured for 1^{st} activation resulting in a displacement or transition of +/−50 mm in the dorsal-ventral direction, or posterior-anterior direction.

The brace may be configured for 2^{nd} activation resulting in a displacement or transition of +/−50 mm in the cranial-caudal direction, i.e. along the proximal axis.

The brace may be configured for 3^{rd} activation resulting in a rotation or flex, dorsal flex, in the sagittal plane of +/−90 degrees.

The brace may be configured for 4^{th} activation resulting in a displacement or transition of +/−15 mm in medial-lateral direction.

The brace may be configured for 5^{th} activation resulting in a rotation about the proximal axis, i.e. the cranial-caudal axis, which may substantially be in the transverse plane, of about +/−90 degrees.

The brace may be configured for 6^{th} activation resulting in a rotation about the distal axis, i.e. substantially a supination or pronation, of +/−90 degrees.

The brace may be configured with a scale display and/or means to record readings of the actual displacement.

A brace may be configured different ranges of rotational angles or linear displacements.

For one or more of the displacement or rotations, the brace may be configured with a scale display and/or means to record readings of the actual displacement/rotation.

In an aspect, the route of displacements and rotations may be recorded. There may be a 1^{st} route comprising a 1^{st} activation of 10 mm, a 2^{nd} activation of −5 mm, and a 6^{th} activation of +20 degrees. There may be a 2^{nd} route with a different order of the same activations, just as an example.

In an aspect the brace is implemented with all six activation means. Thus, the brace will be able to tabulate all possible (six degrees of freedom) displacements and/or rotations.

In example, the brace may be used as follows: One by one torque wrenches are turned to a position just before the ankle is damaged. The positions are kept manually.

All torque wrenches are at the same time forced towards a maximum until the ankle breaks.

In example, the brace may be used as follows: One by one the torque wrenches are turned to a position just before the ankle is damaged. The positions are locked by mechanical systems. Then one chosen torque wrench is forced towards a maximum until the ankle breaks.

### DESCRIPTION OF THE DRAWING

The disclosure is described by example only and with reference to the drawings, whereon:

FIG. 1 illustrates a brace, an anatomical structure and degrees of freedom of the anatomical structure;

FIG. 2 illustrates a brace with activation means, an anatomical structure;

FIG. 3 illustrates braces, each brace capable of rotating or displacing an anatomical structure in a different direction;

FIG. 4 illustrates a 1^{st} activation means of a brace;

FIG. 5 illustrates a 2^{nd} activation means of a brace;

FIG. 6 illustrates part of a proximal part;

FIG. 7 illustrates a brace joint connected to a distal part via a ball joint being a 3^{rd}, 5^{th} and 6^{th} activation means;

FIG. 8 illustrates another embodiment of a brace joint adapted to be equipped with a 3^{rd}, 5^{th} and 6^{th} activation means;

FIG. 9 illustrates further embodiments of a brace joint adapted to be equipped with a 3^{rd}, 5^{th} and 6^{th} activation means;

FIG. 10 illustrates a distal part with a 4^{th} activation means;

FIG. 11 illustrates an embodiment of an i^{th} activation means and a medial-lateral displacement mechanism.

FIG. 12 illustrates a leg of a cadaver placed in a brace;

FIG. 13 illustrates a method for measuring a threshold injury force on an anatomical structure of a human cadaver.

### DETAILED DESCRIPTION

| Item | No |
|---|---|
| Proximal Axis/Cranial-Caudal axis | 10 |
| Proximal end | 12 |
| Distal end | 14 |
| Distal axis/Dorsal-Ventral axis | 20 |
| Dorsal end | 22 |
| Ventral end | 24 |
| Tilt axis/Medial-Lateral axis | 30 |
| Left Lateral end | 32 |
| Medial | 33 |
| Right Lateral end | 34 |
| Sagittal plane | 50 |
| Coronal plane | 60 |
| Transverse plane | 70 |
| Anatomical structure | 90 |
| Proximal anatomical structure | 91 |
| Anatomical joint | 92 |
| Distal anatomical structure | 93 |
| Adjacent anatomical structure | 94 |
| Foot | 95 |
| Leg | 96 |
| Upper leg | 97 |
| Lower leg | 98 |
| Ankle | 99 |
| Brace | 100 |
| 1^{st} Activation means (dorsal-ventral displacement) | 101 |
| 2^{nd} Activation means (cranial-caudal displacement) | 102 |
| 3^{rd} Activation means (flex in sagittal plane) | 103 |
| 4^{th} Activation means (medial-lateral displacement) | 104 |
| 5^{th} Activation means (rotation of distal part | 105 |
| about proximal axis = cranial-caudal axis) | |
| 6^{th} Activation means (rotation of distal | 106 |
| part about distal axis = dorsal-ventral axis) | |
| Torsion spring | 110 |
| Handle | 120 |
| Nut | 130 |
| Proximal part | 200 |
| Proximal joint end | 202 |
| Proximal free end | 204 |
| Proximal upper body | 206 |
| Proximal channel | 207 |
| Proximal lower body | 208 |
| Proximal fixation | 210 |
| Proximal parallel arms | 220 |
| Proximal free end fixation | 230 |
| Proximal guide | 240 |
| Linear rack gear | 242 |
| Slider body | 244 |
| Upper slider body | 245 |
| Lower slider body | 246 |
| Rod | 248 |
| Thread | 249 |
| Brace joint | 300 |
| Ball joint | 310 |
| Brace jacket | 320 |
| Brace jacket arm | 322 |
| Brace inner body | 330 |
| Brace channel | 340 |
| Brace pin | 350 |
| Mid opening | 360 |
| End opening | 370 |
| Inner body pin | 380 |
| Distal Part | 400 |
| Distal joint end | 402 |
| Distal free end | 404 |
| Distal plate | 410 |
| Proximal plate | 420 |
| Distal parallel arms | 430 |
| Distal parallel arms joint end | 432 |
| Distal parallel arms free end | 434 |
| Brace | 500 |
| Human Cadaver | 900 |
| Method for measuring a threshold fracture | 1000 |
| force of an ankle of a human cadaver | |
| Providing | 1100 |
| Securing | 1200 |
| Applying | 1300 |
| Forcing | 1310 |
| Locking | 1320 |
| Measuring | 1400 |

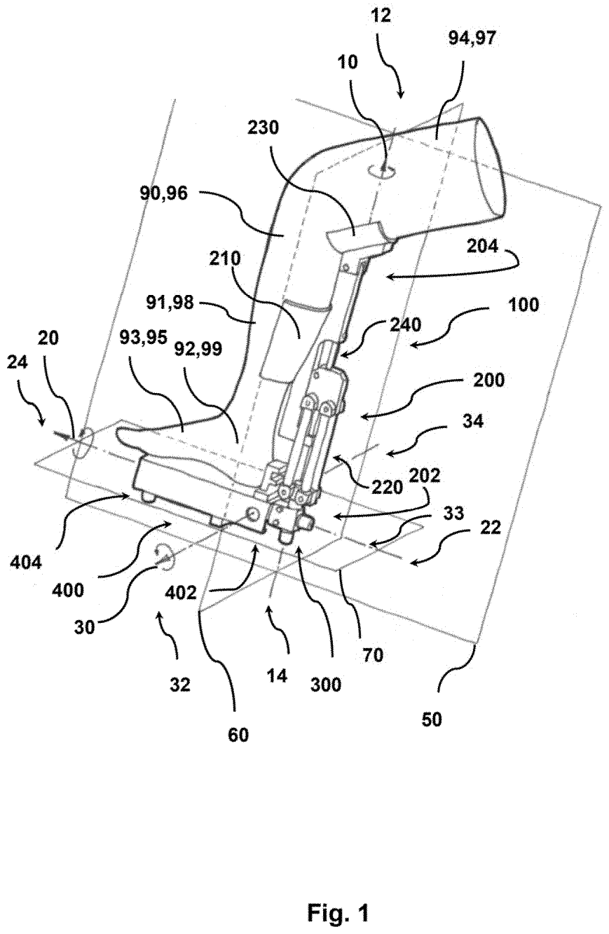

FIG. 1 illustrates a brace 100, an anatomical structure 90 and degrees of freedom of the anatomical structure 90.

The anatomical structure 90 has a proximal anatomical structure 91, a distal anatomical structure 93, an anatomical joint 92 interconnecting the proximal anatomical structure 91 and the distal anatomical structure 93.

The proximal anatomical structure 91 is further connected to an adjacent anatomical structure 94 distal to the distal anatomical structure 93.

The movement of the distal anatomical structure 93 relative to the proximal anatomical structure 91 is controlled by the anatomical joint 92.

The brace 100 has a proximal part 200 extending from a proximal joint end 202 towards a proximal free end 204 along a proximal axis 10.

The proximal part 200 comprises a proximal fixation 210, which is configured for supporting the proximal anatomical structure 91.

The proximal part 200 comprises a proximal free end fixation 230, which is configured for supporting the adjacent anatomical structure 94.

The brace 100 comprises a distal part 400 extending from a distal joint end 402 towards an opposite a distal free end 404 along a distal axis 20.

The distal part 400 is adapted for supporting the distal anatomical structure 93.

The brace 100 comprises a brace joint 300. The proximal part 200 at the proximal joint end 202 interacts via the brace joint 300 with the distal part 400 at the distal joint end 402.

The distal axis 20 has a dorsal end 22 in the direction of the distal joint end 402 and a ventral end 24 opposite to the dorsal end 22. The distal axis 20 may also be called dorsal-ventral axis 20.

The proximal axis 10 has a distal end 14 in a direction of the proximal joint end 202 and a proximal end 12 opposite to the distal end 14 and in the direction of the proximal free end 204. The proximal axis 10 may also be called cranial-caudal axis 10. The proximal axis 10 and the distal axis 20 define a sagittal plane 50.

A tilt axis 30 is perpendicular to both the proximal axis 10 and the distal axis 20 and intersects the distal joint end 402. The part of the tilt axis 30 intersecting the distal joint end 402 is a medial 33 and the ends of the tilt axis 30 are respectively a left lateral end 32 and a right lateral end 34. The tilt axis 30 may also be called medial-lateral axis 30. The tilt axis 30 and the distal axis 20 define a transverse plane 70. The tilt axis 30 and the proximal axis 10 define a coronal plane 60.

The brace 100 comprises a pair of proximal parallel arms 220. The pair of proximal parallel arms 220 are connected to and arranged along the proximal part 200 and operatively connected to the distal part 400 for a displacement of the distal part 400 relative to the proximal part 200 substantially along the distal axis 20. This may be performed by a 1^{st} activation means 10.

The proximal part 200 comprises a proximal guide 240, which is configured for a displacement of the distal part 400 relative to the proximal free end fixation 230 along the proximal axis 10. This may be performed by a 2^{nd} activation means 102. The displacement is a cranial-caudal displacement.

The brace joint 300 is configured to rotate the distal part 400 in the sagittal plane 50 spanned by the proximal axis 10 and the distal axis 20.

The distal part 400 comprises means for displacement of the distal anatomical structure 93 parallel to the tilt axis 30. Thereby, the distal anatomical structure 93 can be displaced towards the left lateral end 32 or the right lateral end 34.

The brace joint 300 is configured to rotate the distal part 400 substantially about the proximal axis 10 in the transverse plane 70. The brace joint 300 is configured to rotate the distal part 400 about the distal axis 20.

Thereby the brace 100 is able to control six degrees of freedom of the anatomical structure 90.

In this specific embodiment, the anatomical structure 90 is a leg 96, the proximal anatomical structure 91 is a lower leg 98, the distal anatomical structure 93 is a foot 95, the anatomical joint 92 is an ankle 99 and the adjacent anatomical structure 94 is an upper leg 97.

The ankle 99 is able to move in the previously described six degrees of freedom and thus to be able to treat a broken ankle 99, it may be necessary to control all six degrees of freedom of the broken ankle to give a proper support of the entire leg 96.

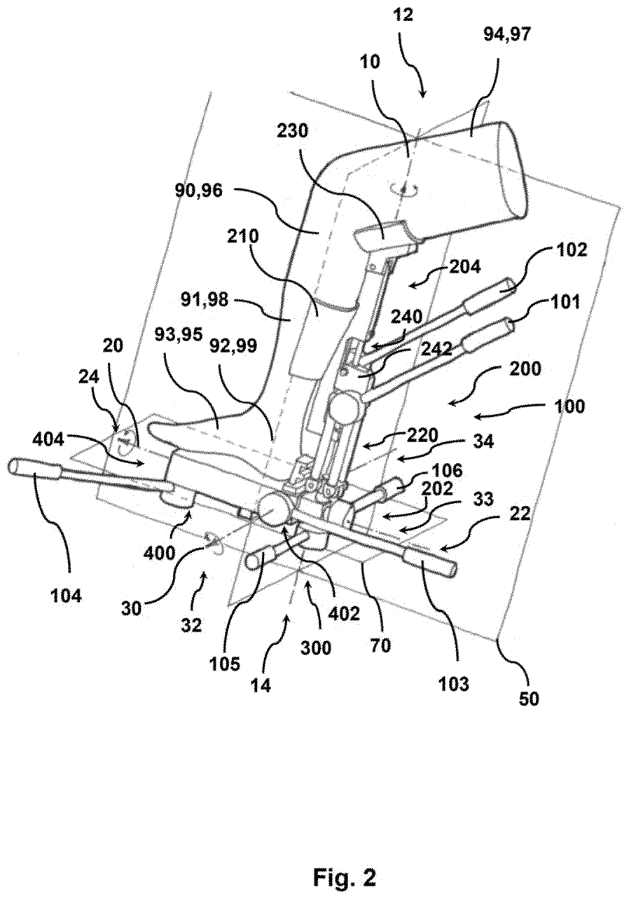

FIG. 2 illustrates a brace 100 with activation means 101, 102, 103, 104, 105, 106 and an anatomical structure 90.

The brace 100 and the anatomical structure 90 have all the same features as the brace 100 and the anatomical structure 90 shown in the FIG. 1. The brace 100 is likewise able to displace and rotate the anatomical structure 90 in and around the axis 10, 20, 30 and planes 50, 60, 70.

The brace 100 comprises a pair of proximal parallel arms 220. The pair of proximal parallel arms 220 are connected to and arranged along the proximal part 200 and operatively connected to the distal part 400 for a displacement of the distal part 400 relative to the proximal part 200 substantially along the distal axis 20. This may be performed by a 1^{st} activation means 101.

The displacement of the distal part 400 along the distal axis 20 is controlled by a 1^{st} activation means 101. The 1^{st} activation means 101 will cause, if activated, the distal part and thus the distal anatomical structure 93, 95 to be displaced towards either the dorsal end 22 or the ventral end 24.

The anatomical joint 92, 99 enables the distal anatomical structure 93, 95 to be displaced towards either the dorsal end 22 or the ventral end 24. Since a torque wrench is the 1^{st} activation means 101, the applied force on the anatomical structure 90, 96 along the distal axis 20 can be controlled precisely and thus the movement of the anatomical joint 92, 99 can be controlled precisely.

The proximal part 200 comprises a proximal guide 240, which is configured for a displacement of the distal part 400 relative to the proximal free end fixation 230 along the proximal axis 10. This may be performed by a 2^{nd} activation means 102. The displacement is a cranial-caudal displacement.

The displacement of the distal part 400 along the proximal axis is controlled by a 2^{nd} activation means 102. The 2^{nd} activation means 102 will cause, if activated, the proximal guide 240 to displace the distal part 400 relative to the proximal free end fixation 230.

The proximal guide 240 comprises a linear rack gear 242 capable of displacing the proximal free end fixation 230 along the proximal axis 10 thereby changing the distance between the distal part 400 and the proximal free end fixation 230.

The 2^{nd} activation means 102 being operatively connected to the linear rack gear 242.

The displacement of the distal part 400 relative to the proximal free end fixation 230 will cause the distal anatomical structure 93, 95 to be displaced relative to the adjacent anatomical structure 94, 97.

The anatomical joint 92, 99 enables the distal anatomical structure 93, 95 to be displaced relative to the adjacent anatomical structure 94, 97 along the proximal axis 10.

Since a torque wrench is the 2^{nd} activation means 102, the applied force on the anatomical structure 90, 96 along the proximal axis 10 can be controlled precisely and thus the movement of the anatomical joint 92, 99 can be controlled precisely.

The brace joint 300 is configured to rotate the distal part 400 in the sagittal plane 50 spanned by the proximal axis 10 and the distal axis 20. The rotation of the distal part 400 in the sagittal plane 50 is operatively controlled by a 3^{rd} activation means 103.

The rotation of the distal part 400 in the sagittal plane 50 causes the distal anatomical structure 93 to either rotate towards the proximal anatomical structure 91, 98 or rotate away from the proximal anatomical structure 91, 98.

The anatomical joint 92, 99 enables the distal anatomical structure 93, 95 to rotate, in the sagittal plane 50, towards and away from the proximal anatomical structure 91, 98.

Since a torque wrench is the 3^{rd} activation means 103, the applied torque on the distal anatomical structure 93, 95 in the sagittal plane 50 can be controlled precisely, and thus the movement of the anatomical joint 92, 99 can be controlled precisely.

The distal part 400 comprises 4^{th} activation means 104 for displacement of the distal anatomical structure 93, 95 parallel to the tilt axis 30. Thereby, the distal anatomical structure 93, 95 can be displaced towards the left lateral end 32 or the right lateral end 34.

The anatomical joint 92, 99 enables the distal anatomical structure 93, 95 to be displaced along the tilt axis 30 towards the left lateral end 32 and the right lateral end 34.

Since a torque wrench is the 4^{th} activation means 104, then the applied force on the distal anatomical structure 93, 95 along the tilt axis 30 can be controlled precisely and thus the movement of the anatomical joint 92, 99 can be controlled precisely.

The brace joint 300 is configured to rotate the distal part 400 substantially about the proximal axis 10 in the transverse plane 70. The brace joint 300 comprises a 5^{th} activation means 105 for controlling the rotation of the distal part 400 in the transverse plane 70 about the proximal axis 10. The anatomical joint 92, 99 enables the distal anatomical structure 93, 95 to be rotated about the proximal axis 10 in the transverse plane 70.

Since a torque wrench is the 5^{th} activation means 105, the applied torque on the distal anatomical structure 93, 95 about the proximal axis 10 in the transverse plane 50 can be controlled precisely and thus the movement of the anatomical joint 92, 99 can be controlled precisely.

The brace joint 300 is configured to rotate the distal part 400 about the distal axis 20. The brace joint 300 comprises a 6^{th} activation means 106 for controlling the rotation of the distal part 400 about the distal axis 20. The anatomical joint 92, 99 enables the distal anatomical structure 93, 95 to be rotated about the distal axis 20.

Since a torque wrench is the 6^{th} activation means 106, then the applied torque on the distal anatomical structure 93, 95 about the distal axis 20 can be controlled precisely and thus the movement of the anatomical joint 92, 99 can be controlled precisely.

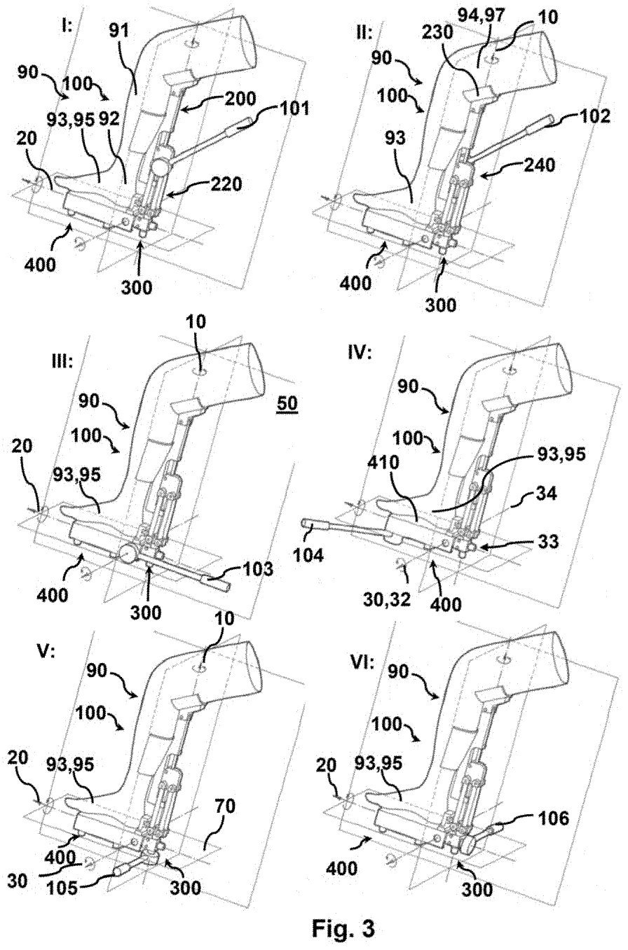

FIG. 3 illustrates braces 100, each brace capable of rotating or displacing an anatomical structure 90 in a different direction.

FIG. 3I illustrates a brace 100 capable of displacing a distal anatomical structure 93 along a distal axis 20.

The brace 100 comprises a proximal part 200 interacting via a brace joint 300 with a distal part 400 extending along the distal axis and being adapted for supporting the distal anatomical structure 93, wherein a pair of proximal parallel arms 220 are connected to and arranged along the proximal part 200 and operatively connected to the distal part 400 for a displacement of the distal part 400 relative to the proximal part 200 substantially along the distal axis 20.

The brace 100 comprises 1^{st} activation means 101 for controlling the displacement along the distal axis 20. The 1^{st} activation means 101 may be a handle or a torque wrench.

The skilled person would know that the brace 100 could be a brace 100 only capable of displacing the distal anatomical structure 93 along the distal axis 20 or a brace 100 capable of rotating or displacing the anatomical structure 90 in different directions, but where all means except the means for displacing the distal anatomical structure 93 along the distal axis 20 are locked in place.

The skilled person would know that when a foot 95 is the distal anatomical structure 93, then the distal axis 20 would be equivalent to the dorsal-ventral axis 10.

FIG. 3II illustrates a brace 100 capable of displacing a distal anatomical structure 93 along a proximal axis 10 relative to an adjacent anatomical structure 94.

The brace 100 comprises a proximal part 200 extending along the proximal axis and interacting via a brace joint 300 with a distal part 400 for supporting the distal anatomical structure 93.

The proximal part 200 further comprises a proximal free end fixation 230 for supporting the adjacent anatomical structure 94 and a proximal guide 240 for displacement of the proximal free end fixation 230 along the proximal axis 10 relative to the distal part 400.

The brace 100 comprises 2^{nd} activation means 101 for controlling the displacement along the proximal axis 10, thereby the distal anatomical structure 93 can be displaced relative to the adjacent anatomical structure 94.

The 2^{nd} activation means 102 may be a handle or a torque wrench.

The skilled person would know that the brace 100 could be a brace 100 only capable of displacing the distal anatomical structure 93 relative to the adjacent anatomical structure 94 along the proximal direction 10 or a brace 100 capable of rotating or displacing the anatomical structure 90 in different directions, but where all means, except the means for displacing the distal anatomical structure 93 relative to the adjacent anatomical structure 94 along the proximal direction 10, are locked in place.

The skilled person would know that when a foot 95 is the distal anatomical structure 93 and an upper leg 97 is the adjacent anatomical structure 94, then the proximal axis 20 would be equivalent to the cranial-caudal axis 20.

FIG. 3III illustrates a brace 100 capable of rotating a distal anatomical structure 93 in a sagittal plane 50 defined by a distal axis 20 and a proximal axis 10.

The brace 100 comprises a brace joint 300 and a distal part 400 extending along the distal axis 20 and being adapted for supporting a distal anatomical structure 93. The brace joint 300 is configured to rotate the distal part 400 in the sagittal plane 50, thereby enabling the brace 100 to rotate a distal anatomical structure 93 in the sagittal plane 50.

The brace joint 300 comprises 3^{rd} activations means 103 for controlling the rotation of the distal part 400 in the sagittal plane 50. The 3^{rd} activation means 103 may be a handle or a torque wrench.

The skilled person would know that the brace 100 could be a brace 100 only capable of rotating the distal anatomical structure 93 in the sagittal plane 50 defined by the distal axis 20 and the proximal axis 10 or a brace 100 capable of rotating or displacing the anatomical structure 90 in different directions, but where all means except the means for rotating the distal anatomical structure 93 in the sagittal plane 50 defined by the distal axis 20 and the proximal axis 10 are locked in place.

FIG. 3IV illustrates a brace 100 capable of displacing a distal anatomical structure 93 along the tilt axis 30 between a medial 33 and a left lateral end 32 or a right lateral end 34.

The brace 100 comprises a distal part 400 having means for displacing a distal plate 410 along the tilt axis 30, thereby enabling the brace 100 to displace a distal anatomical structure 93 along the tilt axis 30 between the medial 33 and the left lateral end 32 or the right lateral end 34.

The brace joint 300 comprises 4^{th} activations means 104 for controlling the displacement of the distal part 400 along the tilt axis 30. The 4^{th} activation means 104 may be a handle or a torque wrench.

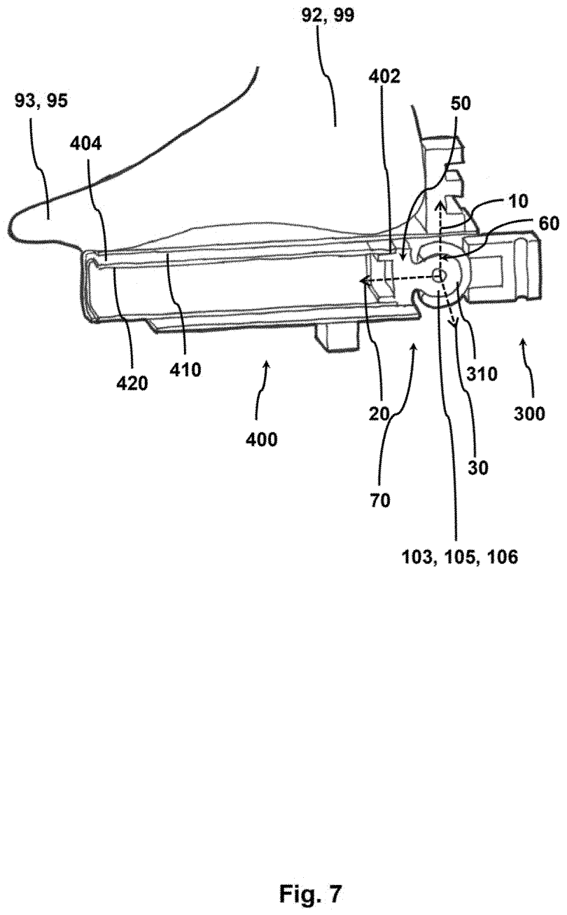

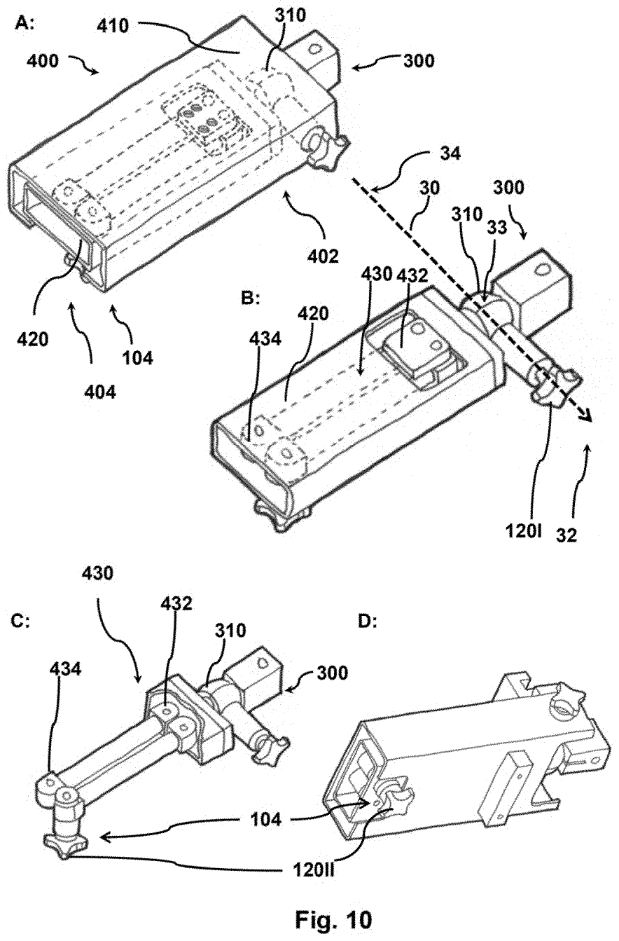

The means for displacing the distal plate 410 are shown in greater detail in subsequent figures e.g. FIGS. 7 and 10.

The skilled person would know that the brace 100 could be a brace 100 only capable of displacing the distal anatomical structure 93 along the tilt axis 30 between the medial 33 and the left lateral end 32 or the right lateral end 34 or a brace 100 capable of rotating or displacing the anatomical structure 90 in different directions, but where all means, except the means for displacing the distal anatomical structure 93 along the tilt axis 30 between the medial 33 and the left lateral end 32 or the right lateral end 34, are locked in place.

The skilled person would know that when a foot 95 is the distal anatomical structure 93, the tilt axis 30 would be equivalent to the medial-lateral axis 30.

FIG. 3V illustrates a brace 100 capable of rotating a distal anatomical structure 93 around a proximal axis 10 in a transverse plane 70.

The brace 100 comprises a brace joint 300 being configured by a 5^{th} activation means 105 to rotate a distal part 400 substantially about the proximal axis 10 in the transverse plane 70. Thereby, the brace 100 is enabled to rotate a distal anatomical structure 93 about the proximal axis 10 in the transverse plane 70.

The 5^{th} activations means 105 controls the rotation of the distal part 400 about the proximal axis 10 in the transverse plane 70. The 5^{th} activation means 105 may be a handle or a torque wrench.

The skilled person would know that the brace 100 could be a brace 100 only capable of rotating the distal anatomical structure 93 around the proximal axis 10 in the transverse plane 70 or a brace 100 capable of rotating or displacing the anatomical structure 90 in different directions, but where all means except the means for rotating the distal anatomical structure 93 around the proximal axis 10 in the transverse plane 70 are locked in place.

The skilled person would know that when a foot 95 is the distal anatomical structure 93, then the proximal axis 10 would be equivalent to the cranial-caudal axis 10 and the transverse plane 70 would be spanned by the dorsal-ventral axis 20 and the medial-lateral axis 30.

FIG. 3VI illustrates a brace 100 capable of rotating a distal anatomical structure 93 about a distal axis 20.

The brace 100 comprises a brace joint 300 being configured by a 6^{th} activation means 106 to rotate a distal part 400 substantially about the distal axis 20, thereby enabling the brace 100 to rotate a distal anatomical structure 93 about the distal axis 20. The 6^{th} activation means 106 may be a handle or a torque wrench.

The skilled person would know that the brace 100 could be a brace 100 only capable of rotating the distal anatomical structure 93 about the distal axis 20 or a brace 100 capable of rotating or displacing the anatomical structure 90 in different directions, but where all means except the means for rotating the distal anatomical structure 93 about the distal axis 20 are locked in place.

The skilled person would know that when a foot 95 is the distal anatomical structure 93, the distal axis 20 would be equivalent to the dorsal-ventral axis 20.

FIG. 2 discloses a brace 100 comprising the 1^{st}-6^{th} activation means 101, 102, 103, 104 105 and 106. The resulting brace 100 is able to control the six rotations or displacements of a anatomical structure 90 at the cost of the brace 100 becoming more complicated.

The purpose of the brace 100 is to treat an anatomical joint 92 interconnecting a proximal anatomical structure 91 and the anatomical distal structure 93. Depending on the trauma of the anatomical joint 92, there may not be needed more than a single displacement or a single rotation for the brace 100 to be able to treat the anatomical joint 92, thus the brace 100 may only have the 1^{st} activation means 101 disclosed in FIG. 3I.

However, it may be necessary to use a brace 100 having the 2^{nd}, 4^{th} and 6^{th} activation means 102, 104, 106 if the trauma of the anatomical joint 92 is more complicated.

Thus, a brace 100 capable of treating a trauma of an anatomical joint 92 may have any combination of features disclosed in FIGS. 3I-3VI.

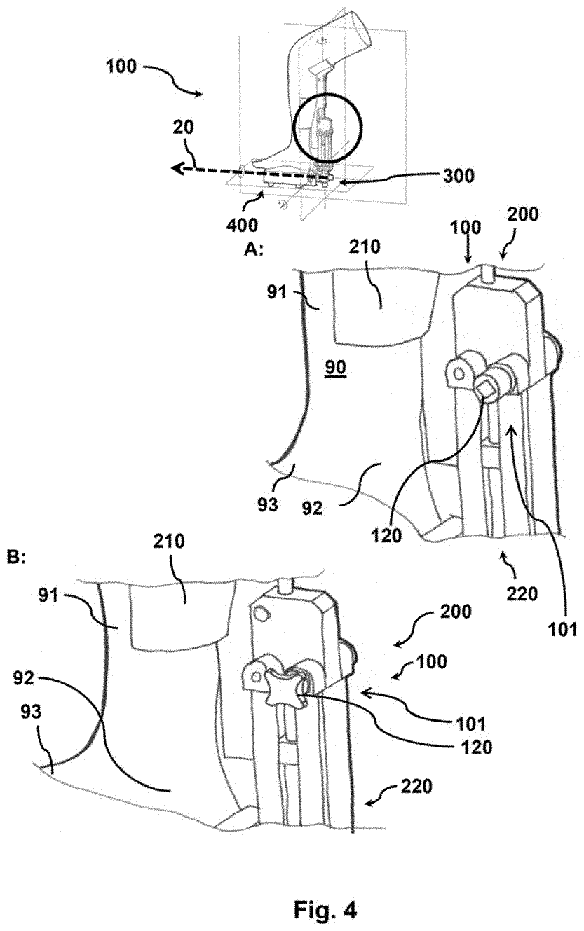

FIG. 4 illustrates a 1^{st} activation means 101 of a brace 100.

The figure shows two different embodiments (A, B) of the 1^{st} activation means 101. FIG. 4A and FIG. 4B both show a part of the brace 100 as shown at the top of FIG. 4.

FIG. 4A discloses an anatomical structure 90 with a proximal anatomical structure 91 connected to a distal anatomical structure 93 via an anatomical joint 92.

The brace 100 comprises a proximal part 200 with a proximal fixation 210 for supporting the anatomical structure 90 and two pairs of proximal parallel arms 220 interacting via a brace joint 300 to a distal part 400 supporting the distal anatomical structure 93.

The two pairs of proximal parallel arms 220 increase the stability of the brace 100 significantly. The two pairs of proximal parallel arms 220 enable displacement of the distal part 400 and thus the distal anatomical structure 93 substantially along a distal axis 20.

The displacement along the distal axis 20 is controlled by the 1^{st} activation means 101 positioned in an arm of the pairs of proximal parallel arms 220. The 1^{st} activation means 101 connected to a handle 120, where a rotation of the handle 120 causes a biasing force, which displaces the distal part 400 supporting the distal anatomical structure 93 substantially along the distal axis 20.

In the embodiment of FIG. 4A, the handle 120 is a female socket for interacting with a torque wrench or the like. FIG. 4B discloses an anatomical structure 90 with a proximal anatomical structure 91 connected to a distal anatomical structure 93 via an anatomical joint 92. The embodiment in FIG. 4B differs only from FIG. 4A by having a different type of handle 120 adapted to be manually rotatable by hand.

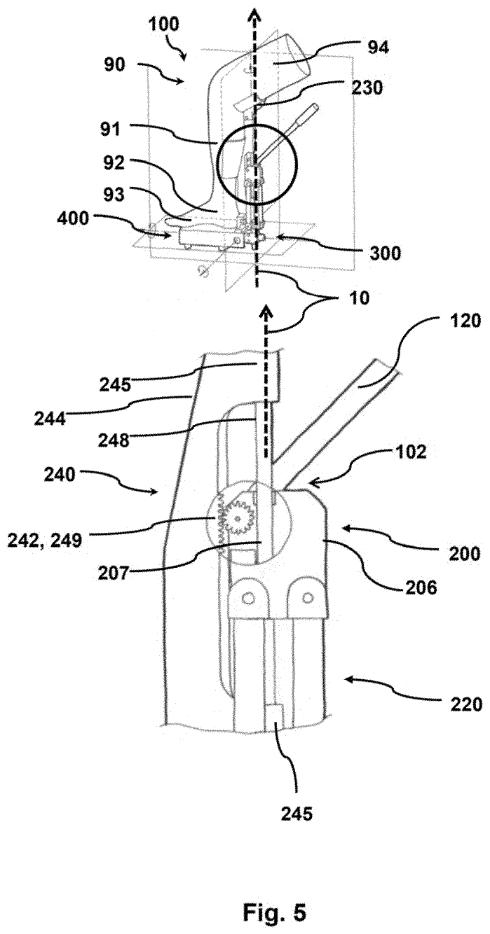

FIG. 5 illustrates a 2^{nd} activation means 102 of a brace 100.

The brace 100 at the top of FIG. 5 supports an anatomical structure 90 with a proximal anatomical structure 91 connected to a distal anatomical structure 93 via an anatomical joint 92. The circle at the brace 100 is shown in close-up.

The close-up discloses a proximal part 200 having a proximal upper body 206 with at least a pair of proximal parallel arms 220 for interaction with a distal part 400 via a brace joint 300.

The proximal upper body 206 has a through-going proximal channel 207 being substantially parallel to a proximal axis 10.

The proximal part 200 further comprises a proximal guide 240. The proximal guide 240 comprises a slider body 244 having an upper slider body 245 and a lower slider body 244, the proximal guide 240 with a rod 248 extending between the upper and lower slider body 245, 246.

The rod 248 being positioned in the proximal channel 207 such that the slider body 244 can only be displaced along the proximal axis 10.

The proximal upper body 206 and the slider body 244 have combined a linear rack gear 249, where the gear is positioned in the proximal upper body 206 facing a side of the slider body 244 between the upper and lower slider body 245, 246, which side of the slider body 244 being threaded for interacting with the gear.

The linear rack gear 249 is the 2^{nd} activation means 102, because the rotation of the rack gear 249 will cause the slider body 244 to be displaced along the proximal axis 10. Thereby the distal plate 400 will be displaced relative to the slider body 244. The linear rack gear 249 may be activated by a handle 120, which could be a torque wrench.

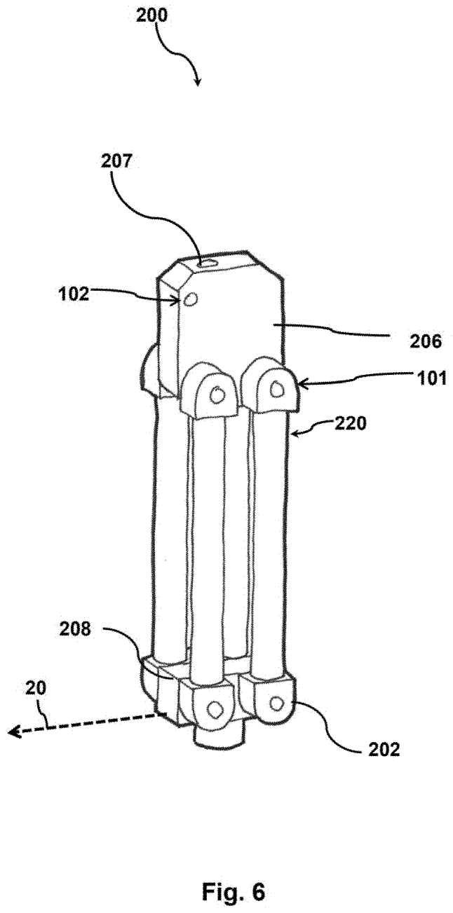

FIG. 6 illustrates part of a proximal part 200 comprising an upper body 206, a lower body 208, and two pairs of parallel arms 220 extending between the upper body 206 and the lower body 208.

Thereby, the lower body 208 can be displaced relative to the upper body 206 substantially along a distal axis 20.

The lower body 208 is the proximal joint end 202 and will translate the displacement relative to the upper body 207 through a brace joint 300 (not shown) to a distal part 400 (not shown).

The displacement can be controlled through the 1^{st} activation means 101.

The upper body 208 comprises a trough going proximal channel 207 for a not shown rod 248 and not shown gear. The gear is part of 2^{nd} activation means 102, shown in more detail in FIG. 5.

FIG. 7 illustrates a brace joint 300 connected to a distal part 400 via a ball joint 310 being a 3rd, 5th and 6^{th} activation means 103, 105 and 106.

The distal part 400 comprises a distal plate 410 connected to a proximal plate 420, which proximal plate 420 is connected to the ball joint 310.

A distal anatomical structure 93 with an anatomical joint 92 is positioned on the distal plate 410. In this specific example the distal anatomical structure 93 is foot 95 and the anatomical joint 92 is an ankle 99.

Although not shown there are means for securing the distal anatomical structure 93, 95 to the distal plate 400 such that the displacement or rotation of the distal plate 410 is transferred to the distal anatomical structure 93, 95 and thus the anatomical joint 92.

The distal part 400 has a distal joint end 402 and a distal free end 402. A distal axis 20 is defined in the direction from the distal joint end 402 to the distal free end 402.

A proximal axis 10 disclosed in the figure, a not shown proximal part 200 would extend along the proximal axis 10 when the distal part 400 and the brace joint 300 is part of a brace 100 according to the disclosure.

A tilt axis 30 is extending through the ball joint 310 while being perpendicular to both the distal axis 20 and the proximal axis 10.

In the present figure the distal anatomical structure 93 is a foot 95 and thus the proximal axis 10 is equivalent to the cranial-caudal axis 10, the distal axis 20 is equivalent to the dorsal-ventral axis 20 and the tilt axis 30 is equivalent to the medial-lateral axis 30.

The proximal axis 10 and the distal axis 20 span a sagittal plane 50. The distal axis 20 and the tilt axis 30 span a transverse plane 70. The tilt axis 30 and the proximal axis 10 span a coronal plane 50.

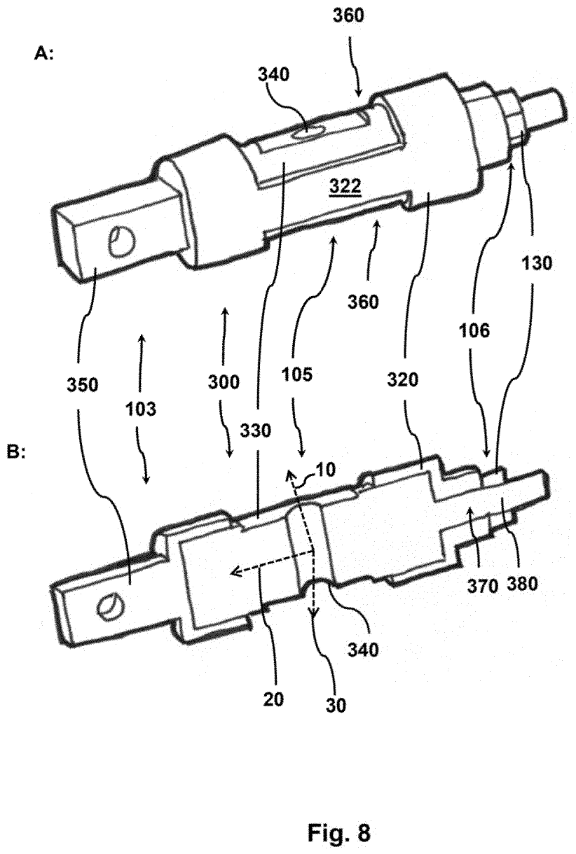

FIG. 8 illustrates another embodiment of a brace joint 300 adapted to be equipped with a 3^{rd}, 5^{th} and 6^{th} activation means 103, 105, 106.

FIG. 8A discloses the brace joint 300 in full and FIG. 8B discloses a cross section of the brace joint 300. FIG. 8B discloses the proximal axis 10, the distal axis 20, and the tilt axis 30.

The brace joint 300 has a brace pin 350, which is to be connected to a distal part 400 (not shown) by a nut 130 (not shown). Thereby the not shown nut 130 will function as the 3^{rd} activation means 103. Thereby the distal part 400 may be displaced and locked in position, relative to the proximal part, in the plane defined by the proximal axis 10 and the distal axis 20, e.g. the distal part 400 may be rotated around the tilt axis 30, e.g. relative to the proximal part.

The brace joint 300 comprises a brace jacket 320 encompassing a brace inner body 330, where the brace inner body 330 rotational displaceable in the brace jacket 320. For example, the brace jacket 320 surrounds or encloses at least part of the brace inner body 330.

The brace jacket 320 has near the brace pin 350 a first U-shaped end completely surrounding a first end of the brace inner body 330 and distal to the first U-shaped end a second U-shaped end completely surrounding a part of the brace inner body 330. The second U-shaped end having an aperture, e.g. an end opening (370), through which a second end, e.g. an inner body pin (380), of the brace inner body 330 extends.

A nut 130 engages with a threaded part of the second end, e.g. the inner body pin (380), of the brace inner body 330 and when tightened a friction force between the nut 130 and the brace jacket 320 will prevent rotation around the distal axis 20 between the brace inner body 330 and the brace jacket 320. For example, the nut 130 may be turned into a tightened position and a loosened position. In the tightened position the nut 130 abuts the brace jacket 320, such that there is no room for movement between the nut 130 and the brace jacket 320 keeping the brace inner body 330 from rotating relative to the brace jacket 320. The nut 130 may be turned into a loosened position. In the loosened position the brace inner body 330 is free to rotate around the distal axis 20 relative to the brace jacket 320. Thereby the nut 130 is the 6^{th} activation means 106.

The brace jacket 320 further comprises two brace jacket arms 322, but only one is shown. The brace jacket arms 322 connect the U-shaped ends of the brace jacket 320 while exposing two surfaces of the brace inner body 330. The brace jacket 320 comprises one or more, such as two, mid openings 360, e.g. formed by the U-shaped ends and the two brace jacket arms 322. The mid openings 360 allows access to the brace inner body 330. The brace inner body 330 has a brace channel 340 going through the brace inner body 330 from the one surface to the other surface. The brace channel 340 enable a not shown distal part 400 to be rotated relative to a not shown proximal part 200 around the proximal axis 10. Part of the proximal part 200 may extend through the brace channel 340. For example, the proximal part 200 may comprise a pin configured to extend through the brace channel 340. When the proximal part 200 extends through the brace channel 340 the rotation of the brace inner body 330 relative to the brace jacket 320 is restricted by the interaction between the proximal part 200 and the mid openings 360 and/or brace jacket arms 322.

Depending on the overall design one or two nuts on one or both sides of the brace channel 340, respectively, will be the 5^{th} activation means 105.

Thereby, the brace joint 300 is adapted to be equipped with a 3^{rd}, 5^{th} and 6^{th} activation means.

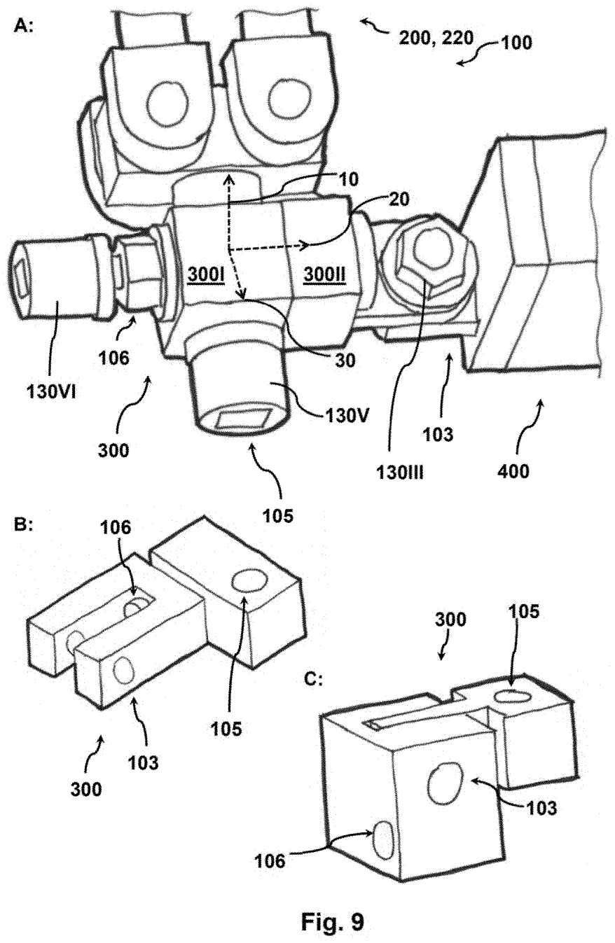

FIG. 9 illustrates further embodiments of a brace joint 300 adapted to be equipped with a 3^{rd}, 5^{th} and 6^{th} activation means 103, 105, 106.

FIG. 9A discloses a section of a brace 100 near the brace joint 300. The brace joint 300 is connected to a proximal part 200 with proximal parallel arms 220, and a distal part 400. FIG. 9A further discloses the proximal axis 10, the distal axis 20, and the tilt axis 30.

The brace joint 300 comprises a nut 130III at the connection between the brace joint 300 and the distal part 400. The distal part 400 becomes pivotal in the plane defined by the proximal axis 10 and the distal axis 20, i.e. the sagittal plane, when the nut 130III is loosened. The distal part 400 becomes locked in the sagittal plane when the nut 130III is tightened. Thus, the nut 130III is 3^{rd} Activation means 103.

The brace joint 300 comprises a nut 130V controlling the connection between the brace joint 300 and the proximal part 200. The loosening of the nut 130V enabled the distal part 400 to be rotationally displaced relative to the proximal part 200 around the proximal axis 10. The tightening of the nut 130VI locks the distal part 400 in a specific position. Thus, the nut 130V is 5^{th} activation means 105.

The brace joint 300 is in two parts 300I, 300II at least partially relative to each other. The brace joint 300 comprises a nut 130VI controlling the friction between the two parts 300I, 300II. The second part 300II of the brace joint 300 is connected to the distal part 400, while the first part 300I of the brace joint 300 is connected to the proximal part 200.

The loosening of nut 130VI will enable the two parts 300I, 300II to rotate relative to each other, thereby the distal part 400 is rotated relative to the proximal part 200 around the distal axis. The tightening of nut 130VI will lock the position of the two parts 300I, 300II relative to each other, thereby the distal part 400 is locked to the proximal part 200 in a position relative to the distal axis. Thus, the nut 130VI is 6^{th} activation means 106.

FIG. 9B-9C discloses two brace joints 300, which both are adapted to be equipped with a 3^{rd}, 5^{th} and 6^{th} activation means 103, 105, 106. Both embodiments in FIG. 9B-9C can replace the brace joint 300 in FIG. 9A.

The skilled person would be able to design other brace joints 300, which are adapted to be equipped with a 3^{rd}, 5^{th} and 6^{th} activation means 103, 105, 106.

FIG. 10 illustrates a distal part 400 capable of medial-lateral displacement.

FIGS. 10A-D disclose the same distal part 400 with different parts visible, where FIG. 10A discloses a double see-through view of the distal part 400, FIG. 10B discloses the distal part 400 without a distal plate 410, FIG. 10C discloses the distal part without the proximal plate 420, and FIG. 10d discloses the distal part 400 from a bottom view.

A ball joint 310 of a brace joint 300 is connected to the distal plate 400. The functions of the ball joint 310 are described the description of FIG. 7. The position of the ball joint 310 can be locked using a handle 1201. Alternatively, the brace joint 300 of FIGS. 8-9 may replace the ball joint 310.

The distal part 400 comprises a distal joint end 402 near the ball joint 310 and an opposite distal free end 404.

The distal part 400 comprises a distal plate 410 for positioning of a distal anatomical structure 93 (not shown) such as a foot 95 (not shown), a proximal plate 420 connected to the ball joint 310, and a pair of distal parallel arms 430, e.g. two arms 430, interconnecting the proximal plate 420 with the distal plate 410.

The pair of distal parallel arms 430 having a distal parallel arms joint end 432 connected to the distal plate 410 near the distal joint end 402 through a bore in the proximal plate 420, and a distal parallel arms free end 434 connected to proximal plate 420 near the distal free end 404.

The positioning of the pair of distal parallel arms 430, and thus the distal plate 410, relative to the proximal plate 420, is controlled by 4^{th} activation means 104. The 4^{th} activation means may be tightened or loosened.

The pair of distal parallel arms 430 enables the distal plate 410 to be displaced along a tilt axis 30 between a medial 33 position towards either a left lateral end 32 or a right lateral end 34. When the 4^{th} activation means is loosened the distal parallel arms 430 are free to displace the distal plate relative to the proximal plate along the tilt axis. When the distal plate has obtained the desired position the 4^{th} activation means may be tightened to retain the obtained position.

The pair of distal parallel arms 430 may alternatively be one arm.

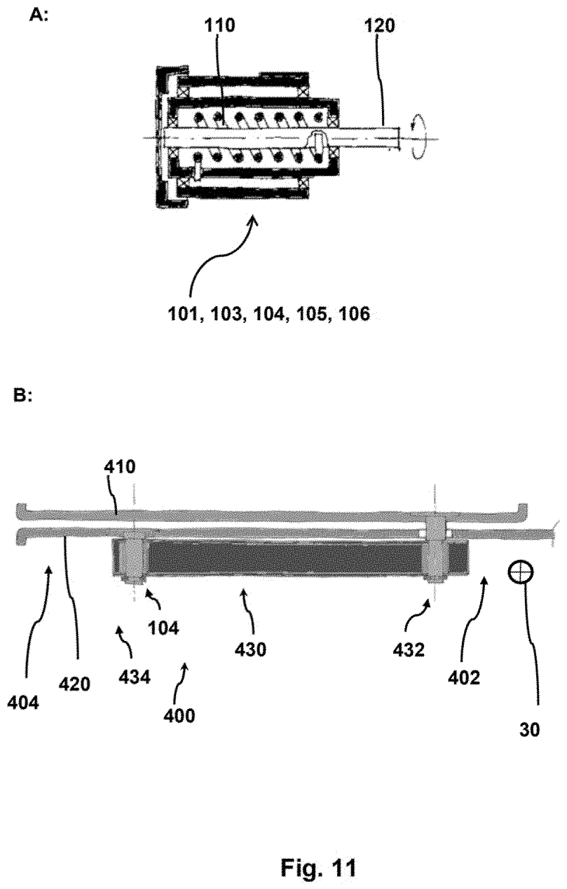

FIG. 11 illustrates an embodiment (A) of an i^{th} activation means 101, 103, 104, 105, 106 and a medial-lateral displacement mechanism (B).

FIG. 11A illustrates an embodiment of the activation means 101, 103, 104, 105, 106 comprising a housing containing a torsion spring 110 connected to a handle 120. The rotation of the handle 120 will be transferred to the torsion spring 110 causing a biasing force causing a part of a brace 100 (not shown) to be displaced or rotated.

FIG. 11B illustrates the medial-lateral displacement mechanism of a distal part 400. The distal part 400 has a distal joint end 402 and an opposite distal free end 404.

The distal part 400 comprises a distal plate 410 for placement of an anatomical structure 90, a proximal plate 420 for connection with a not shown brace joint 310 at the distal joint end 402, and a pair of distal parallel arms 430 interconnecting the proximal plate 420 with the distal plate 410 through a bore in the proximal plate 420.

The pair of distal parallel arms 430 are positioned below the proximal plate 420 and have a distal parallel arms free end 434 connected to the proximal plate 420 in the direction of the distal free end 404 and an opposite distal parallel arms joint end 432 connected to the distal plate 410 through the bore in the proximal plate 420.

The bore having a size allowing the distal plate 410 to be displaced relative proximal plate 420 along a tilt axis 30.

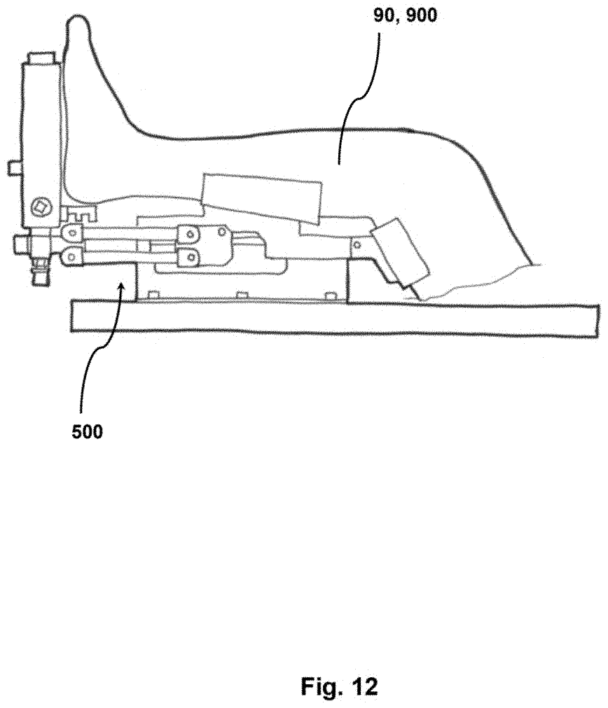

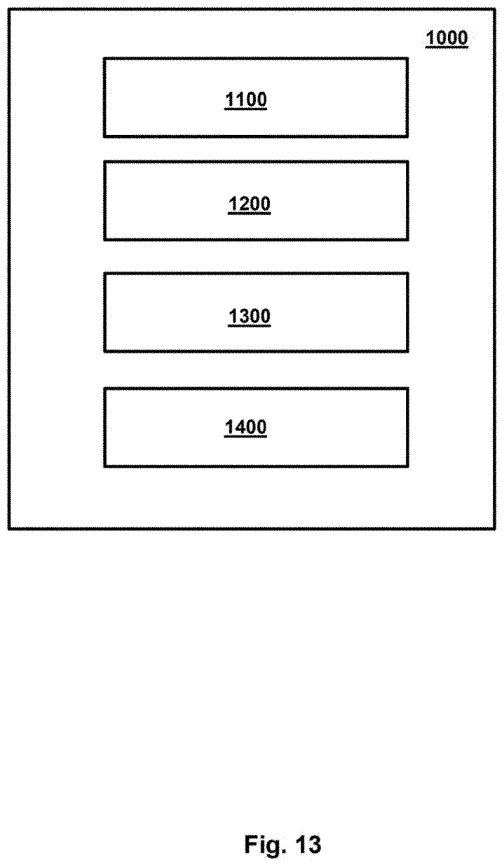

FIG. 12 illustrates a brace 500 for measuring a threshold fracture force, e.g. threshold injury force, of a joint, e.g. an ankle, of an anatomical structure 90 of a human cadaver 900. The brace 500 of FIG. 12 may have the same features as the brace of the previous figures. The method for measuring a threshold fracture force is described in relation to FIG. 13.

FIG. 13 illustrates a method 1000 for measuring a threshold fracture force, e.g. threshold injury force, of an anatomical structure, such as an ankle, of a human cadaver.

The method 1000 comprises providing 1100 a human cadaver having an anatomical structure. The anatomical structure may comprise a lower leg, an ankle, and a foot.

The method 1000 comprises arranging 1200 a brace, such as the brace as described in relation to the previous figures, about an anatomical structure. The anatomical structure may be fixed to the brace with one or more fasteners. For example, part of the foot may be fixed to a distal part of the brace and the lower leg may be fixed to a proximal part of the brace.

The method 1000 comprises applying 1300 one or more forces to the anatomical structure by use of the brace until a point of injury of the anatomical structure. For example, the foot may be rotated relative to the lower leg until the anatomical structure is injured, e.g. in the ankle joint. The force may be applied with a torque wrench and/or a handle. The required torque will depend on the species and in the case of a human, whether the cadaver is a cadaver of a man, a woman, or a child. The required torque also depends on the individual's size.

Applying 1300 one or more forces may comprise turning the torque wrenches one by one to a position just before the anatomical structure is damaged. This position may be kept manually. Alternatively, the positions are locked by a mechanical system.

All torque wrenches may at the same time be forced towards a maximum until the anatomical structure breaks. Alternatively, one chosen torque wrench may be forced towards a maximum until the anatomical structure breaks.

The method 1000 comprises measuring 1400 the one or more forces and/or displacements until and/or at the point of injury. The brace may be configured with a scale display and/or means to record readings of the actual displacement/rotation.

Thereby is achieved a way of tabulating forces and displacements leading to an injury.

Items

1. A brace (100) for management of an injured part of an anatomical structure (90), the brace (100) comprising:
a proximal part (200) extending from a proximal joint end (202) towards a proximal free end (204) along a proximal axis (10) and comprising a proximal fixation (210) configured for supporting a proximal anatomical structure (91); the proximal part (200) at the proximal joint end (202) interacting via
a brace joint (300) with
a distal part (400) extending from a distal joint end (402) towards an opposite distal free end (404) along a distal axis (20),

wherein
a pair of proximal parallel arms (220) are connected to and arranged along the proximal part (200) and operatively connected to the distal part (400) for a displacement of the distal part (400) relative to the proximal part (200) substantially along the distal axis (20).

2. The brace (100) according to item 1, wherein the brace joint (300) is configured with locking means for locking the brace joint (300) and placing the proximal part (200) and the distal part (400) in a fixed position relative to each other and the brace joint (300) is configured to receive gauge means for gauging a force on and/or a dis-placement of the proximal part (200) and the distal part (400) relative to each other.

3. The brace (100) according to item 1 or 2, wherein the proximal part (200) supports a proximal free end fixation (230) configured for supporting an adjacent anatomical structure (97); and the proximal part (200) supports a proximal guide (240) being con-figured for a displacement of the distal part (400) relative to the proximal free end fixation (230) along the proximal axis (10).

4. The brace (100) according to one or more preceding items, wherein the brace joint (300) is configured to rotate the distal part (400) in a plane spanned by the proximal axis (10) and the distal axis (20).

5. The brace (100) according to one or more preceding items, wherein the distal part (400) comprises a distal plate (410) operatively connected to a proximal plate (420) via a pair of distal parallel arms (430) for a displacement of the proximal plate (420) relative to the distal plate (430).

6. The brace (100) according to one or more preceding items, wherein the brace joint (300) is configured to rotate the distal part (400) substantially about the proximal axis (10).

7. The brace (100) according to one or more preceding items, wherein the brace joint (300) is configured to rotate the distal part (400) about the distal axis (20).

8. A trauma brace (500) for injuring an anatomical structure (90) of a human cadaver (900), wherein the trauma brace (500) is a rigid version of the brace (100) according to one or more of items 1-7.

9. A method (1000) for measuring a threshold injury force on an anatomical structure (90) of a human cadaver (900), the method (1000) comprising one or more acts of:
providing (1100) a human cadaver (900) having an anatomical structure (90),
arranging (1200) a trauma brace (500) about an anatomical structure (90),
applying (1300) one or more forces to the anatomical structure (90) by use of the trauma brace (500) until a point of injury of the anatomical structure (90), and
measuring (1400) the one or more forces and/or displacements until and/or at the point of injury.

10. The method (1000) according to item 9, wherein the trauma brace (500) is according to item 8.

1-16. (canceled)

17. A brace for management of an injured part of an anatomical structure, the brace comprising
a proximal part extending from a proximal joint end towards a proximal free end along a proximal axis and comprising a proximal fixation configured for supporting a proximal anatomical structure; the proximal part at the proximal joint end interacting via
a brace joint with
a distal part extending from a distal joint end towards an opposite distal free end along a distal axis,
wherein the distal part comprises a distal plate operatively connected to a proximal plate via two distal parallel arms for a displacement of the proximal plate relative to the distal plate, such that the distal plate is displaced along a tilt axis perpendicular to both the proximal axis and the distal axis.

18. The brace according to claim 17 comprising two proximal parallel arms are connected to and arranged along the proximal part and operatively connected to the distal part for a displacement of the distal part relative to the proximal part substantially along the distal axis.

19. The brace according to claim 17, wherein the brace joint is configured with locking means for locking the brace joint and placing the proximal part and the distal part in a fixed position relative to each other.

20. The brace according to claim 17, wherein the brace joint is configured to receive gauge means for gauging a force on and/or a displacement of the proximal part and the distal part relative to each other.

21. The brace according to claim 20, wherein the gauge means is a torque wrench configured to the releasably attached to the brace joint.

22. The brace according to claim 17, wherein the proximal part supports a proximal free end fixation configured for supporting an adjacent anatomical structure; and the proximal part supports a proximal guide being configured for a displacement of the distal part relative to the proximal free end fixation along the proximal axis.

23. The brace according to claim 17, wherein the brace joint is configured to rotate the distal part in a plane spanned by the proximal axis and the distal axis.

24. The brace according to claim 17, wherein the brace joint is configured to rotate the distal part about the tilt axis.

25. The brace according to claim 17, wherein the brace joint is configured to rotate the distal part substantially about the proximal axis.

26. The brace according to claim 17, wherein the brace joint is configured to rotate the distal part about the distal axis.

27. The brace according to claim 17, wherein the brace joint comprises:
a brace jacket comprising a mid opening,
a brace inner body enclosed by the brace jacket and being configured to be rotational displaceable in the brace jacket around the distal axis,
and wherein the mid opening provides access to the brace inner body.

28. The brace according to claim 27, wherein the brace jacket comprises a first end comprising a brace pin configured to be connected to the distal part.

29. The brace according to claim 27, wherein the brace jacket comprises a second end opposite the first end, the second end comprising an end opening, and wherein the brace inner body comprises an inner body pin configured to extend through the end opening.

30. The brace according to claim 27, wherein the brace inner body is configured to be connected to the proximal part through the mid opening.

31. The brace according to claim 27, wherein the brace inner body comprises a brace channel configured to receive part of the proximal part, the proximal part being configured to be rotational displaceable relative to the brace joint in the brace channel.

32. A method for measuring a threshold injury force on an anatomical structure of a human cadaver, the method comprising one or more acts of:
providing a human cadaver having an anatomical structure,
arranging a brace according to claim 17 about an anatomical structure,
applying one or more forces to the anatomical structure by use of the brace until a point of injury of the anatomical structure, and
measuring the one or more forces and/or displacements until and/or at the point of injury.
